(19) United States
(12) Patent Application Publication
(10) Pub. No.: US 2025/0017980 A1
(43) Pub. Date: Jan. 16, 2025

(54) METHODS OF TREATMENT UTILIZING CANINE-SPECIFIC THERAPEUTIC COMPOSITIONS

(71) Applicant: AlphaLogix, LLC, Phoenix, AZ (US)

(72) Inventors: Christian Beaudry, Phoenix, AZ (US); Jaehyun Kim, Phoenix, AZ (US)

(21) Appl. No.: 18/899,395

(22) Filed: Sep. 27, 2024

Related U.S. Application Data
(63) Continuation-in-part of application No. 18/056,145, filed on Nov. 16, 2022, now Pat. No. 12,128,073.
(60) Provisional application No. 63/280,024, filed on Nov. 16, 2021.

(51) Int. Cl. A61K 35/50; A61K 35/16; A61K 35/19; A61K 35/51; A61P 35/00

(52) U.S. Cl. CPC A61K 35/50; A61K 35/16; A61K 35/19; A61K 35/51; A61P 35/00

(57) ABSTRACT

Methods of treating conditions (e.g., cancer) in a subject using a canine-specific therapeutic composition are described herein. The canine-specific therapeutic composition is clear, safe, and physiologically and biologically active. Methods described herein may comprise administering a therapeutic amount of the canine-specific therapeutic composition which may comprise a micronized canine-specific allantoamnion membrane, in a solution. Thus, the present invention features canine-specific therapeutic compositions comprising placental-derived materials and methods of use.

### CROSS-REFERENCES TO RELATED APPLICATIONS

This application is a continuation-in-part and claims benefit of U.S. patent application Ser. No. 18/056,145 filed Nov. 16, 2022, which is a non-provisional and claims benefit of U.S. Provisional Application No. 63/280,024 filed Nov. 16, 2021, the specifications of which are incorporated herein in their entirety by reference.

### FIELD OF THE INVENTION

The present invention features canine-specific therapeutic compositions comprising placental-derived materials and methods of use.

### BACKGROUND OF THE INVENTION

Dogs are members of the group of species with zonary placentas, meaning that multiple placentas are contained within the uterus. Each placenta is comprised of two distinct sacs, the chorioallantoic sac which is the outer sac containing allantoic fluid, and the amnion sac which is the inner sac containing the amniotic fluid and fetus. The placentas are attached to the uterus wall at the hematophagus zones (marginal hematomas). These are bands of maternal hemorrhage at the margins of the zonary placenta. The products of hemoglobin breakdown give a distinct green tissue coloration. Upon detachment of the placentas from the uterine wall, a dark green discharge (sometimes called uteroverdin) is produced which stains the placentas dark green which makes the tissue very problematic for processing.

This dark green color partially represents the presence of contaminants, particularly blood-related contaminants. The presence of blood-related contaminants, indicated by dark green coloration, may trigger alloimmunization responses in canine allograft recipients, thus harming the recipient and/or causing graft failure, decreasing or negating the therapeutic effects of the allograft materials. Removal of contaminants, especially blood-related contaminants, evidenced in part by the removal of dark green coloration, is a desirable outcome that has not yet been achieved in the prior art but is achieved by the present invention. Removal of contaminants allows genetically non-identical canine patient-recipients to receive allografts more safely and effectively, as the risk of alloimmunization reaction and graft rejection is reduced or eliminated.

The present invention has developed tissue recovery and tissue processing protocols where placentas are recovered during live births or scheduled c-sections, shipped under controlled temperature, and processed into a clear liquid biological product substantially free of contaminants, especially blood-related contaminants, that can be safely administered in dogs, including dogs that are not genetically identical to the donor (i.e., allograft recipients). The tissue processing protocol includes methods for processing and cleaning the incoming dark green placentas to produce a clear amnion injectable product while maintaining its physiological and biological properties.

### BRIEF SUMMARY OF THE INVENTION

It is an objective of the present invention to provide compositions and methods that allow for the production of a clear, safe, and physiologically and biologically active liquid injectable product (i.e., a canine-specific therapeutic composition) for use as a regenerative product in dogs with joint diseases, soft tissue lesions, inflammatory diseases, respiratory diseases, immunological diseases, cancer, neurological diseases, scarring, burns, wounds, eye ulcers, nerve injuries, muscle tears, organ diseases, among others, as specified in the independent claims. The present invention may also be administered via other routes of administration in other embodiments, for example, topically. Embodiments of the invention are given in the dependent claims. Embodiments of the present invention can be freely combined with each other if they are not mutually exclusive.

In some embodiments, the present invention features a method of treating conditions in a subject. In some embodiments, the method comprises administering a therapeutic amount of the canine-specific therapeutic composition comprising a micronized canine-specific allantoamnion membrane in a solution.

In some embodiments, the composition further comprises a micronized canine-specific chorioallantois membrane, micronized canine-specific umbilical cord, micronized canine-specific Wharton's jelly, or a combination thereof. Alternatively, in some embodiments, the composition further comprises micronized canine-specific allantoamnion membrane particles, micronized canine-specific chorioallantois membrane particles, micronized canine-specific umbilical cord particles, micronized canine-specific Wharton's jelly particles, or a combination thereof. In some embodiments, the composition further comprises canine-specific amniotic fluid, canine-specific allantoic fluid, canine-specific stem cells, or a combination thereof. In some embodiments, the compositions described herein may comprise proteins, exosomes, or a combination thereof.

In some embodiments, the composition is substantially free of blood-related contaminants and/or uteroverdin. In some embodiments, the composition is combined or mixed with stem cells prior to administration. Alternatively, or in addition to, in some embodiments, the composition is combined or mixed with platelet plasma protein solutions prior to administration.

Non-limiting examples of conditions that may be treated with the canine-specific therapeutic composition described herein include but are not limited to, inflammatory diseases, joint diseases, tendon and/or ligament injuries, and pain (e.g., pain associated with hip dysplasia, osteoarthritis, or rheumatoid arthritis).

In further embodiments, the present invention features a method of treating a target site in a subject. The method may comprise administering a therapeutic amount of the canine-specific therapeutic composition comprising a micronized canine-specific allantoamnion membrane, in a solution, to the treatment location in the subject. Non-limiting examples of target sites include but are not limited to a wound, an operative incision, an articular joint, a muscle, soft tissue, spine or spinal fluid, an eye and ophthalmic structures, an artery, a vein, a tumor, or a pulmonary airway.

In some embodiments, the present invention features canine-specific therapeutic composition comprising a micronized canine-specific allantoamnion membrane, in a solution. In some embodiments, the composition is substantially free of blood-related contaminants and/or uteroverdin. In some embodiments, the composition further comprises a micronized canine-specific chorioallantois membrane, micronized canine-specific umbilical cord material, amniotic fluid, allantoic fluid, or a combination thereof

In other embodiments, the present invention may further feature a method of producing a canine-specific therapeutic composition. In some embodiments, the method comprises obtaining a whole canine placenta. In some embodiments, the method comprises dissecting the whole canine placenta and retaining an allantoamnion membrane. In some embodiments, the method comprises rinsing the allantoamnion membrane and drying the allantoamnion membrane. In some embodiments, the method comprises micronizing the aforementioned allantoamnion membrane and resuspending the micronized allantoamnion membrane into a working solution to create a canine-specific therapeutic composition. In other embodiments, the method further comprises freezing the canine-specific therapeutic composition to preserve proteins. In other embodiments, the method further comprises freeze-drying the canine-specific therapeutic composition.

One of the unique and inventive technical features of the present invention is the retention and use of all layers in the allantoamnion membrane. Without wishing to limit the invention to any theory or mechanism, it is believed that the technical feature of the present invention advantageously provides for a canine-specific therapeutic composition comprising a high concentration of proteins. The human fetal membrane is derived from the inner and outer layers of a single amniotic sac and is comprised of two conjoined membranes-amnion and chorion. The amnion faces the fetus, and the chorion faces the uterus. In dogs, the amnion membrane is part of the allantoamnion sac, which contains the fetus and amniotic fluid and the chorion membrane is part of the chorionallantois sac, which contains the allantoic fluid. The histological representation of the canine allantoamnion membrane is distinct from the human amnion membrane. The allantoamnion membrane comprises an epithelium layer and a basement membrane layer on both sides of a stromal layer. The allantoamnion membrane composition comprises cells, cellular excretions, cellular derivatives, and extracellular matrix components. The allantoamnion membrane is not attached to the chorionallantois, unlike the human amnion and chorion membranes. Therefore, canine-specific therapeutic compositions described herein are distinct and unique. None of the presently known prior references or work has the unique inventive technical feature of the present invention. For example, there are no other therapeutic compositions that utilize the allantoamnion membrane specifically from a canine.

Another of the unique and inventive technical features of the present invention is the ability to not only retain and use all layers in the allantoamnion membrane, but to do so while also substantially removing immunogenic contaminants. Removal of immunogenic contaminants, particularly blood-related contaminants, is accompanied by creation of a substantially clear and colorless product, including upon suspension in a working solution. Without wishing to limit the present invention to any particular theory or mechanism, it is believed that the technical feature of the present invention advantageously provides for a high concentration of proteins while removing components that may elicit an alloimmunization response when introduced into a canine patient. Blood group antigens are one of the major classes of alloantigens. Multiple potentially immunogenic contaminants, including blood-derived contaminants are advantageously removed with the present invention. A byproduct of this process is that the final allograft product of this invention is a colorless, clear or cloudy suspension, as compared to prior art approaches, which have been unable to produce a product substantially free of the characteristic dark green color of the canine placenta. As this dark green color is the result of the breakdown of bilirubin, a product of heme catabolismthe production of a colorless product is a byproduct of the present invention's inventive feature that allows the removal of immunogenic contaminants, especially blood-related contaminants. Without wishing to limit the present invention to any particular theory or mechanism, it is believed that the technical feature of the present invention therefore provides for increased safety and efficacy by reducing the chance that a recipient of the canine allograft develops an alloimmune response. Safety is increased as alloimmune responses can result in transfusion reactions, graft rejection, and other harmful adverse effects that may harm the canine patient directly. Furthermore, efficacy is also increased, as the likelihood of graft rejection is greatly reduced, therefore increasing the chance that the allograft is able to exert a therapeutic effect on the canine patient-recipient.

Furthermore, the inventive technical features of the present invention contributed to a surprising result. For example, the present invention does not utilize a transport solution (i.e., a saline solution) to transport the whole canine placenta obtained. Surprisingly, it was discovered that approximately 40% more proteins are retained in the membrane of the placenta when transported without a transport solution (i.e., a saline solution). Without wishing to limit the present invention to any particular theory or mechanism, it is believed that the technical feature of the present invention, therefore provides for greater efficacy by maintaining a greater amount of usable protein in the allograft product than previous approaches. Additionally, prior references teach away from the present invention as others utilize a saline solution to transport placentas and/or amniotic membrane pieces.

Additionally, the inventive technical features of the present invention contributed to another surprising result. For example, the Inventors surprisingly found that the transformative process of micronization significantly changes the natural form and properties of the placenta tissue. As the amniotic membrane is micronized into micron-sized particles, its original characteristics (e.g., serve as a protective barrier safeguarding the developing fetus during gestation) are fundamentally altered. This alteration extends beyond mere physical changes and encompasses modifications to its structural integrity, tensile strength, and elasticity. Moreover, the surface area of the membrane is also affected by micronization. Consequently, the micronized membrane acquires enhanced properties, expanding its potential applications in therapeutic contexts.

Micronization plays a crucial role in controlling and optimizing protein release kinetics for tailored therapeutic requirements. For instance, smaller particles, such as nanoparticles characterized by a higher surface area-to-volume (SA/V) ratio, can be formulated for immediate release. SA/V ratio determines the rate of (protein) release in that SA/V ratio increases as a result of micronization, and this causes more molecules to be exposed on the newly created surface, leading to a faster release.

Nevertheless, micronization presents a delicate process. Achieving a consistent range of particle sizes each time requires meticulous optimization of parameters such as cycles per second, grinding time, cooling times, and more, tailored specifically to therapeutic requirements. Surprisingly, the Inventors have managed to overcome these challenges, obtaining a composition capable of producing both a burst and sustained release of proteins (see FIGS. 12 and 13).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows a methodology of creating a canine-specific therapeutic composition described herein.

FIG. 2A shows a canine placenta and the different sacs and fluids.

FIG. 2B and FIG. 2C shows the histology (FIG. 2B) and schematic view (FIG. 2C) of the amniotic membrane (allantoamnion) in dogs. EL: epithelial layer, BL: basal layer, ML: mesenchymal layer (i.e., stromal layer).

FIG. 3 shows the different layers of the human amniotic membrane.

FIG. 4A shows an incoming canine placental tissue prior to processing with the present invention.

FIG. 4B shows isolation of the allantoamnion sac from the whole canine placenta.

FIG. 4C shows the allantoamnion membrane after processing with the present invention.

FIG. 4D shows canine-specific allantoamnion clear liquid finished product produced with and of the present invention.

FIG. 5 shows that approximately 40% more proteins are retained in the allantoamnion membrane of the placenta when transported without a transport solution, according to the methods of the present invention, as compared to when the placenta is transported with a transport solution.

FIGS. 6A, 6B, and 6C show a canine chest wound before and after treatment with a canine-specific therapeutic composition in accordance with the present invention. FIG. 6A shows a canine chest wound on the day of subcutaneous injection with 2.0 mL of a canine-specific therapeutic composition. FIGS. 6B and 6C show a canine chest wound 9 (FIG. 6B) and 14 (FIG. 6C) days after subcutaneous injection with 2.0 mL of a canine-specific therapeutic composition of the present invention.

FIGS. 7A, 7B, 7C, and 7D show a canine abdominal thermal wound before and after treatment with a canine-specific therapeutic composition in accordance with the present invention. FIG. 7A shows a canine abdominal thermal wound before injection with 2.0 mL of the canine-specific therapeutic composition. FIG. 7B shows a canine abdominal thermal wound on the day of subcutaneous injection with 2.0 mL of the canine-specific therapeutic composition. FIGS. 7C and 7D show a canine abdominal thermal wound 2 (FIG. 7C) and 8 (FIG. 7D) weeks after subcutaneous injection with 2.0 mL of the canine-specific therapeutic composition.

FIGS. 8A, 8B, and 8C show a canine calcanean tendon lesion before and after treatment with a canine-specific therapeutic composition in accordance with the present invention. FIG. 8A shows a canine calcanean tendon lesion before injection with 1.0 mL of a canine-specific therapeutic composition of the present invention. FIGS. 8B and 8C show a canine calcanean tendon lesion 1 month (FIG. 8B) and 2 months (FIG. 8C) after injection with 1.0 mL of a canine-specific therapeutic composition of the present invention.

FIGS. 9A, 9B, and 9C show an acral lick dermatitis lesion before and after treatment with a canine-specific therapeutic composition in accordance with the present invention. FIG. 9A shows an acral lick dermatitis lesion before injection of 1.0 mL of the canine-specific composition. FIGS. 9B and 9C show an acral lick dermatitis lesion 14 days (FIG. 9B) and 2 months (FIG. 9C) after injection of the canine-specific composition.

FIGS. 10A, 10B, and 10C show a squamous cell carcinoma before and after treatment with a canine-specific therapeutic composition in accordance with the present invention. FIG. 10A shows a squamous cell carcinoma lesion before injection of 2.0 mL of the canine-specific composition. FIGS. 10B and 10C show a squamous cell carcinoma lesion 13 (FIG. 10B) and 81 days (FIG. 10C) days after injection of the canine-specific composition.

FIGS. 11A, 11B, and 11C shows flow cytometry analysis of exosomes contained within one sample of the canine-specific composition. FIG. 11A shows a side scatter and forward scatter plot showing the exosome population based on size. FIG. 11B shows a scatter plot showing the exosome population based on positive staining. FIG. 11C shows a table of exosome concentration in 3 separate samples of the canine-specific composition.

FIG. 12 shows that the micronization of membrane into particles has a significant means of controlling release kinetics so to optimize protein release that meets dose for specific therapeutic needs. A study conducted by the Inventors shows how controlling particle size can generate therapeutically effective doses. A cumulative release of a canine-specific composition comprising allantoamnion particles reveals a burst release of proteins equal to 0.34 mg/mL, whereas samples that consist of the intact membrane (natural form) of equal mass, did only 0.04 mg daily up to day 3, which is unlikely to be therapeutically effective. Protein release was 8× greater at day 1 in the micronized membrane samples compared to the intact membrane samples and the increased release was still sustained at day 10.

FIG. 13 shows cumulative protein release from allantomanion particles within the canine-specific composition. Mean cumulative in vitro release of total soluble proteins from allantoamnion particles in the finished product (i.e., the canine-specific composition described herein). The finished product was incubated at 37° C. for 34 days, and protein release from the particles was measured at different time points using a spectrophotometer. An initial release of a total protein was observed at day 1, then a slight increase in cumulative release was sustained up to day 34.

FIG. 14 shows non-limiting examples of proteins and their concentration that may be within the canine-specific therapeutic composition described herein after processing (e.g., contained in the finished product). In some embodiments, the canine-specific therapeutic compositions described herein may comprise alpha-2-macroglubulin (A2M), interleukin-1 receptor antagonist (IL-1ra), interleukin-8 (IL-8), tissue inhibitor of metalloproteinases 2 (TIMP-2), trappin-2, decorin, Cystatin-C, Resistin, epidermal growth factor receptor (EGFR), monocyte chemoattractant protein-1 (MCP-1/CCL2), vascular endothelial growth factor (VEGF), or a combination thereof.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

Placental tissue, as used herein, refers to tissues derived from a placenta, including amnion membrane (i.e., allantoamnion), chorion membrane (i.e., chorioallantois), Wharton's Jelly, umbilical cord material, amniotic fluid, allantoic fluid, and the like.

As used herein, “allantoamnion,” “amnion membrane,” “allantoamnion membrane,” and “amnion” can be used interchangeably and refer to the inner sac comprising the amniotic fluid and fetus.

As used herein, “chorioallantois,” “chorion membrane,” “chorioallantois membrane,” and “chorion” can be used interchangeably and refer to the outer sac comprising the allantoic fluid.

Micronized placental tissue particles, as used herein, are defined as particles derived from placenta, including the allantoamnion, the chorioallantois, the umbilical cord or the Wharton's Jelly. Allantoamnion particles may be preferred for therapeutic effectiveness. Placental tissue may be micronized to have an average particle size of about 100 μm in length, width, or thickness, and is preferably micronized to have an average particle size of about 10 μm in length, width, or thickness. In some embodiments, the placental tissue may be micronized to have an average particle size of about 2000 μm, or about 1800 μm, or about 1500 μm, or about 1250 μm, or about 1000 μm, or about 900 μm, or about 800 μm, or about 700 μm, or about 600 μm, or about 500 μm, or about 450 μm, or about 400 μm, or about 350 μm, or about 300 μm, or about 250 μm, or about 200 μm, or about 150 μm, or about 100 μm, or about 75 μm, or about 50 μm, or about 25, or about 10 μm, or about 5 μm, or about 1 μm, or about 0.5 μm, or about 0.1 μm and any range between and including the average particle sizes provided. Particle size, average particle size, or particle size distribution may be determined by analysis of scanning electron micrographs, or other suitable methods. Micronized placental tissue particles may be formed through any suitable method including but not limited to, tissue grinding, cryogenic fracturing, application of heat and pressure, sonication and/or enzyme digestion. The resulting particles may be either used wet, partially dehydrated or essentially dehydrated by any means known to one of skill in the art such as, for example, lyophilization.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a condition, disease or disorder. The term “patient” includes human and veterinary subjects.

As used herein, a “disease” or “condition” may be used interchangeably and may refer to an abnormal state that impairs the structure or function of part or all of the body, typically characterized by distinct signs and symptoms. Diseases or conditions may include ailments, disorders, illnesses, pain, infections, injuries, allergies, cancer, or the like.

The terms “treating” or “treatment” refer to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; disease modification; healing, or improving a patient's physical or mental well-being.

The terms “manage,” “managing,” and “management” refer to preventing or slowing the progression, spread, or worsening of a disease or disorder, or of one or more symptoms thereof. In certain cases, the beneficial effects that a subject derives from a prophylactic or therapeutic agent do not result in a cure of the disease or disorder.

The term “effective amount” as used herein refers to the amount of a pharmaceutical, therapy, or medication which is sufficient to reduce and/or ameliorate the severity and/or duration of a given disease, disorder, or condition and/or a symptom related thereto. This term also encompasses an amount necessary for the reduction or amelioration of the advancement or progression of a given disease (e.g., cancer), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” is an amount sufficient enough to provide a therapeutic benefit in the treatment or management of a disease, disorder, condition, or to delay or minimize one or more symptoms associated with the presence of a given disease, disorder, or condition. A therapeutically effective amount of an agent means an amount of therapeutic agent, alone or in combination with other therapies, which provides a therapeutic benefit in the treatment or management of the disease, disorder, or condition. The term “therapeutically effective amount” can encompass an amount that improves overall therapy, reduces or avoids symptoms or causes of a given disease, disorder, condition, or enhances the therapeutic efficacy of another therapeutic agent.

The terms “administering”, and “administration” refer to methods of providing a pharmaceutical, therapy, or medication preparation to a subject. Such methods are well known to those skilled in the art and include, but are not limited to, administering the compositions orally, intranasally, parenterally (e.g., intravenously and subcutaneously), by intramuscular injection, intra articular injection, intraligamentary injection, intratendon injection, by intraperitoneal injection, intrathecally, transdermally, extracorporeally, inhalation, topically or the like.

The term “protein” as used herein can be the full length polypeptide, or a fragment or segment of a polypeptide, and can encompass a stretch of amino acids residues of at least about 8 amino acids, generally at least 10 amino acids, more generally at least 20 amino acids, often at least 30 amino acids, more often at least 50 amino acids or more of the full length polypeptide.

The term “exosomes” as used herein are membrane bound extracellular vesicles that contain cellular components, including proteins, growth factors, cytokines, DNA, RNA, lipids, and the like.

As used herein, “contaminants” may refer to substances that are unintentionally introduced into the products or systems, potentially causing harm or undesirable effects. In some embodiments, contaminants may be chemical, biological or physical substances that, when present, make the compositions herein impure, unsafe or unsuitable for intended use.

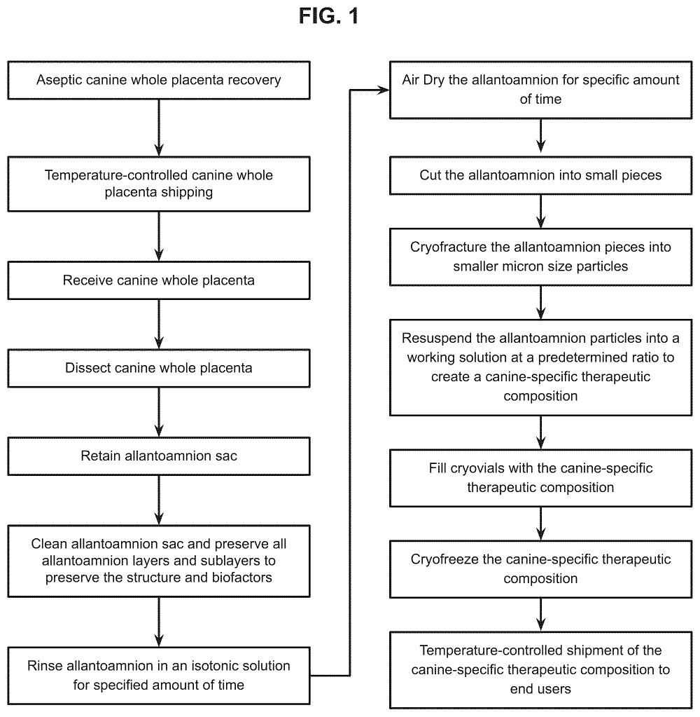

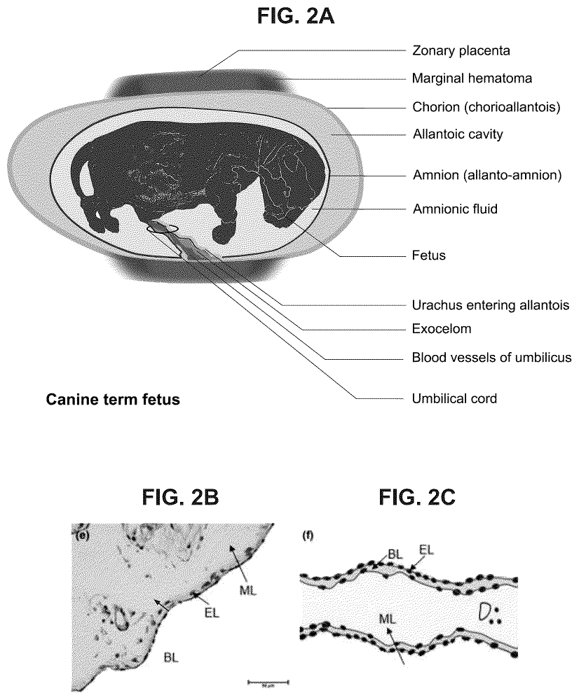

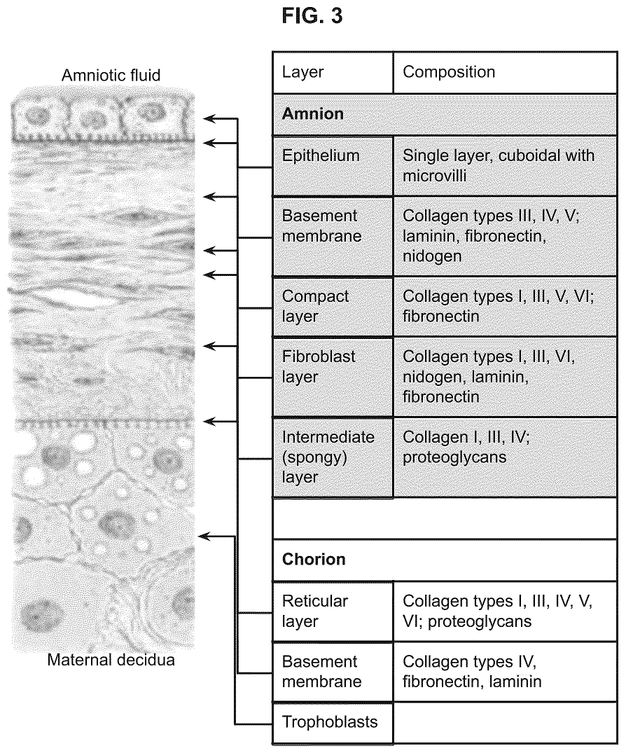

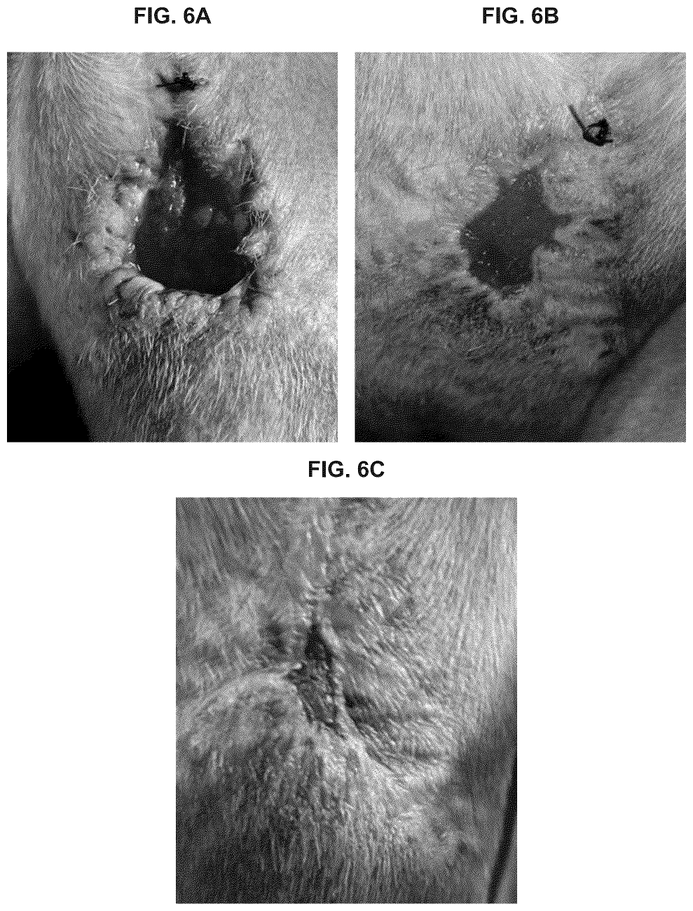

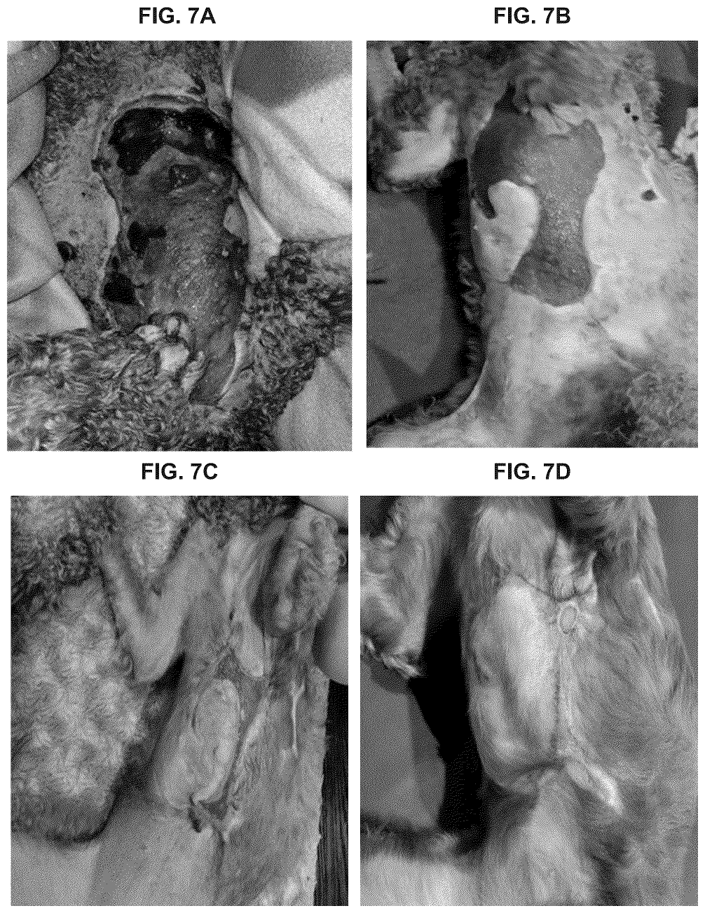

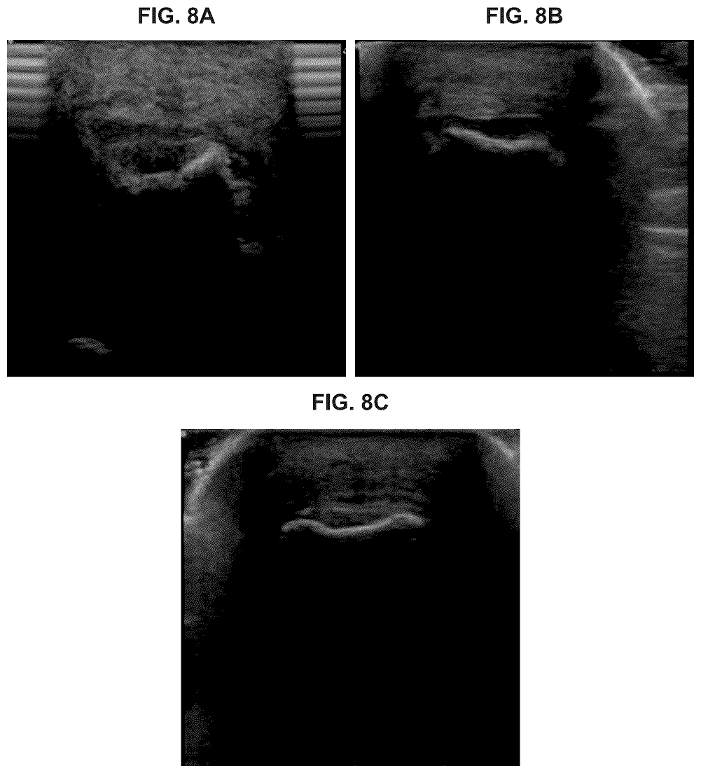

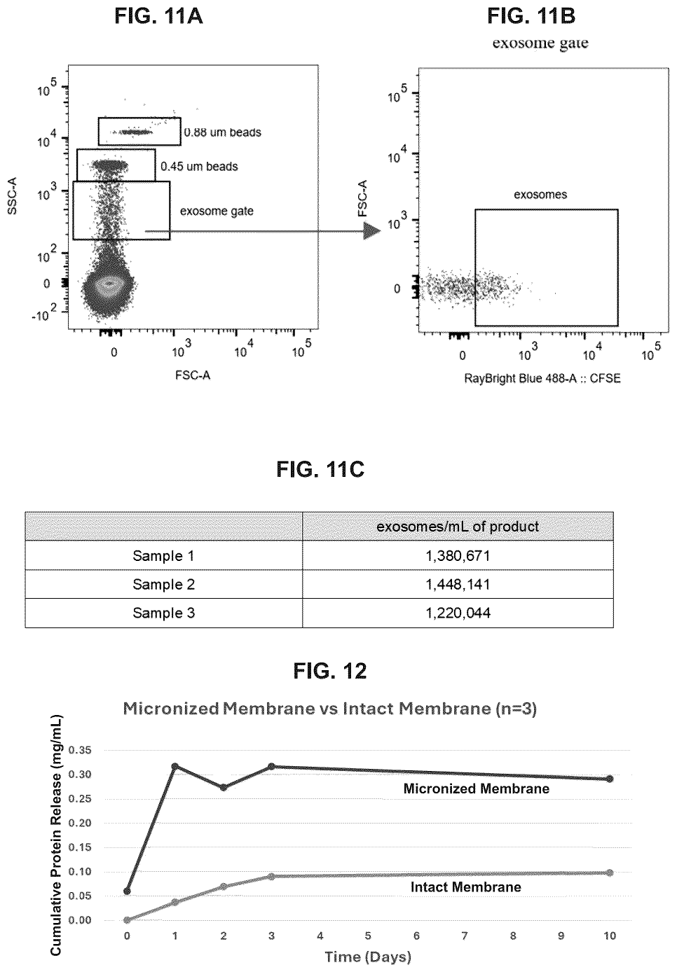

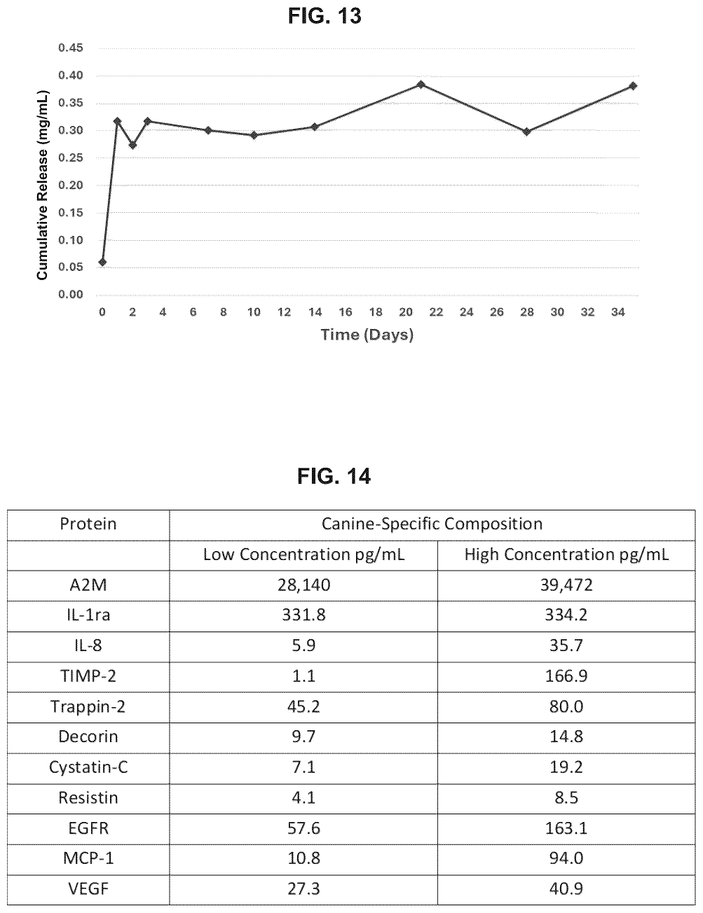

Referring now to FIGS. 1-14, the present invention features methods for processing canine placental tissues into a clear, safe, and physiologically and biologically active liquid product that may be delivered via injection or any other therapeutically appropriate route of administration. Additionally, the present invention features methods for treating animal (e.g., canine) ailments using the canine-specific therapeutic composition produced and described here.

### Canine-Specific Therapeutic Composition

The present invention features a canine-specific therapeutic composition comprising a micronized canine-specific allantoamnion membrane, in a solution. In some embodiments, the composition is substantially free of blood-related contaminants and/or uteroverdin. In some embodiments, the composition may further comprise a micronized canine-specific chorioallantois membrane, micronized canine-specific umbilical cord, micronized canine-specific Wharton's Jelly or a combination thereof. In other embodiments, the composition may further comprise micronized canine-specific allantoamnion membrane particles, micronized canine-specific chorioallantois membrane particles, micronized canine-specific umbilical cord particles, micronized Wharton's Jelly particles, or a combination thereof.

Alternatively, in some embodiments, the present invention may feature a canine-specific therapeutic composition comprising a micronized canine-specific allantoamnion membrane in a solution; wherein the composition is free of blood-related contaminants and/or uteroverdin. Or, in some embodiments, the present invention may feature an injectable canine-specific therapeutic composition comprising a micronized canine-specific allantoamnion membrane; wherein the composition is substantially free of blood-related contaminants and/or uteroverdin.

In some embodiments, the canine-specific therapeutic composition comprises an allantoamnion membrane (e.g., canine-specific allantoamnion membrane), in a working solution. Alternatively, in other embodiments, the canine-specific therapeutic composition comprises allantoamnion membrane particles (e.g., canine-specific allantoamnion membrane particles), in a working solution.

Other placental tissue from any mammalian donor may be used in accordance with the methods and compositions as described herein.

The compositions described herein may also include canine-specific amniotic fluid, allantoic fluid, or a combination of both. Additionally, or alternatively, these compositions may incorporate canine-specific amniotic fluid cells, allantoic fluid cells, cord blood cells, mesenchymal stem cells, or any combination thereof.

The compositions described herein may also be combined or mixed with cells and stem cells from a secondary source. The cells and stem cells may be derived from the blood, bone marrow, adipose tissue, dental pulp, or stromal vascular fraction. In some embodiments, the secondary source may be distinct from placental or donor tissue.

The compositions described herein may also be combined or mixed with platelet rich plasma, platelet poor plasma, serum solutions, or the like.

In some embodiments, the compositions described herein may be combined with platelet-rich plasma, platelet-poor plasma, serum solutions, or similar substances just prior to administration. For example, a lyophilized composition may be reconstituted and then mixed with these substances. Alternatively, the composition may be mixed with platelet-rich plasma, platelet-poor plasma, or serum solutions before lyophilization.

As used herein, a “working solution” or a “solution” may be used interchangeably and may refer to a solution comprising Plasma-Lyte™ A, saline, or sodium phosphate. The present invention is not limited to the solutions described above and may encompass any other suitable solutions known in the art. In other embodiments, the working solution may also comprise NaCl solution, a phosphate buffer, any other isotonic solutions, hypertonic solutions, DMEM, water, or a combination thereof. In further embodiments, the working solution may further comprise a cryopreservative, a stabilizer, buffers, collagen, hyaluronic acid, antimicrobial agents such as antibiotics or antifungal agents, surfactants, pH modifiers, proteins, exosomes, cell fragments, cellular excretions, cellular derivatives, extracellular matrix components, amniotic fluid, allantoic fluid or a combination thereof.

In certain embodiments, the canine-specific therapeutic composition comprises placental tissue (e.g., canine-specific placental tissue) in a working solution. In some embodiments, the placental tissue comprises an allantoamnion membrane, a chorioallantois membrane, an umbilical cord, Wharton's Jelly, or a combination thereof. Thus, in some embodiments, the composition may comprise an allantoamnion membrane, a chorioallantois membrane, an umbilical cord, Wharton's Jelly, or a combination thereof. In some embodiments, the allantoamnion membrane comprises amniotic fluid. In some embodiments, the chorioallantois membrane comprises allantoic fluid.

In some embodiments, the aforementioned composition may further comprise allantoamnion membrane particles, chorioallantois membrane particles, umbilical cord particles, Wharton's Jelly particles or a combination thereof. In other embodiments, the compositions described herein further comprise amniotic fluid, allantoic fluid, or a combination thereof. In other embodiments, the compositions described herein further comprise cells from the amniotic fluid, allantoic fluid, cord blood or a combination thereof.

The allantoamnion consists of three distinct layers: a first epithelial and basement membrane layer, a second mesenchymal (stromal) layer, and a third epithelial and basement membrane layer. In some embodiments, in the compositions described herein, all three layers of the allantoamnion may be utilized, or any combination of the first, second, or third layers may be selected. The mesenchymal layer comprises a matrix of blood vessels, which may be removed to suit specific applications. In some embodiments, blood vessels may be extracted from the mesenchymal layer using forceps, by plucking, or by separating the epithelial and basal layers and peeling off the vessels. The separated epithelial and basal layers (ei.g., the first layer and the third layer) may then be reassembled.

Thus, In some embodiments, the canine-specific compositions described herein are free of blood vessels. In other embodiments, the compositions described herein are substantially free of blood vessels. In further embodiment, the compositions described herein comprises blood vessels. Without wishing to limit the present invention to any theory or mechanism it is believed that the removal of the blood vessels mitigates the risk of a patient having an immunogenic response towards the composition.

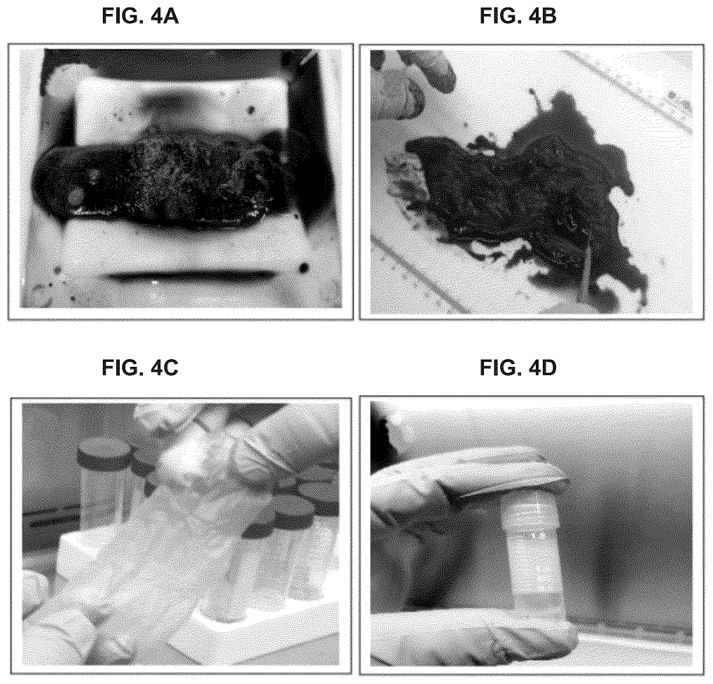

Without wishing to limit the present invention to any theory or mechanism it is believed that within the field it is well established that canine placenta exhibits a distinctive dark green color, attributed to the breakdown of bilirubin from heme catabolismsee FIGS. 4A and 4B). This green coloration, associated with blood-related contaminants and/or uteroverdin, poses a risk of alloimmunization reactions in allograft recipients, potentially compromising graft success and therapeutic effectiveness. Therefore, the removal of these contaminants to achieve a colorless composition is a highly sought-after objective among experts in the field. Accordingly, the present invention features a composition that is substantially free of blood-related contaminants and/or uteroverdin, characterized by its colorless appearance. This distinct characteristic serves as a hallmark within the field, ensuring the composition's suitability for therapeutic applications.

Canine-specific compositions described herein may be free or substantially free of contaminants. In some embodiments, the contaminants are blood-related components. In some embodiments, the contaminants are immunogenic blood components. In some embodiments, the contaminants are uteroverdin. In some components, the contaminants are products of hemoglobin breakdown. In some embodiments, the contaminants are red blood cells. In some embodiments, the contaminants are immunogenic. In some embodiments, the contaminants are immunogenic blood-related components. Without wishing to limit the present invention to any theory or mechanism it is believed that the removal of contaminants mitigates the risk of a patient having an immunogenic response towards the composition, thereby improving patient recipient safety and increasing the effectiveness of the compositions by preventing graft rejection.

In some embodiments, compositions described herein do not elicit an immune-mediated reaction upon introduction into an allograft recipient. In some embodiments, compositions described herein do not elicit a clinically significant immune-mediated reaction upon introduction into an allograft recipient. In some embodiments, compositions described herein do not elicit an alloimmunization response upon introduction into an allograft recipient. In some embodiments, compositions described herein do not elicit a clinically significant alloimmunization response upon introduction into an allograft recipient. An allograft recipient may encompass any of the subjects described herein, such as a canine, feline, or equine recipient, among others. Without wishing to limit the present invention to any theory or mechanism it is believed that these properties of the present invention mitigate the risk of a patient having an immunogenic and/or alloimmunization response towards the composition, thereby improving the patient/recipient safety and increasing the effectiveness of the compositions by preventing graft rejection.

In some embodiments, compositions described herein do not elicit an immune-mediated reaction upon introduction into a xenograft recipient. In some embodiments, compositions described herein do not elicit a clinically significant immune-mediated reaction upon introduction into a xenograft recipient. In some embodiments, compositions described herein do not elicit an alloimmunization response upon introduction into a xenograft recipient. In some embodiments, compositions described herein do not elicit a clinically significant alloimmunization response upon introduction into a xenograft recipient. A xenograft recipient may encompass any of the subjects described herein, such as a canine, feline, or equine recipient, among others.

In some embodiments, compositions described herein are liquids or suspensions substantially free of green color. In some embodiments, the compositions are a substantially colorless liquid or suspension. In some embodiments, the compositions are a substantially clear liquid or suspension. In some embodiments, the compositions are a substantially clear and colorless liquid or suspension. In some embodiments, the compositions are a substantially cloudy and colorless liquid or suspension. Without wishing to limit the present invention to any theory or mechanism it is believed that these properties of the present invention represent removal of contaminants from the compositions of the present invention, thus mitigating the risk of a patient having an immunogenic and/or alloimmunization response towards the composition, thereby improving patient-recipient safety and increasing the effectiveness of the compositions by preventing graft rejection. In some embodiments, the compositions further comprise about 2 cm² of allantoamnion membrane per about 1 mL of working solution. Without wishing to limit the present invention to any theory or mechanism, it is believed that this ratio may produce fewer adverse effects and achieve superior therapeutic results compared to other ratios.

Without wishing to limit the present invention to any theory or mechanism, and referring to FIG. 14, it is believed that amnion is a rich source of biologically active factors involved in tissue regeneration with reported anti-inflammatory, anti-fibrotic, and immunomodulatory properties. The use of canine amniotic tissue allografts to treat conditions in dogs with soft tissue injuries, orthopedic injuries, cancer, and chronic wounds may be minimally invasive, safe, and offer a therapeutic result. Thus, in some embodiments, the compositions described herein comprise proteins. In some embodiments, the proteins are derived from the allantoamnion membrane, an chorioallantois membrane, an umbilical cord, cord blood, Wharton's Jelly or a combination thereof. In some embodiments, the proteins have anti-inflammatory properties, antifibrotic properties, antibacterial properties, anti-fungal properties, anti-viral properties, healing properties, repairing properties, immunoregulatory properties, anti-cancer properties, or a combination thereof.

In some embodiments, the compositions described herein comprise exosomes.

In some embodiments, the exosomes are derived from the allantoamnion membrane, an chorioallantois membrane, an umbilical cord, cord blood, Wharton's Jelly or a combination thereof. In some embodiments, the exosomes have anti-inflammatory properties, antifibrotic properties, antibacterial properties, anti-fungal properties, anti-viral properties, healing properties, repairing properties, immunoregulatory properties, anti-cancer properties, or a combination thereof. In some embodiments, the compositions described herein are clear, safe, and physiologically and biologically active.

Referring to FIG. 14, in some embodiments, the proteins may include but are not limited to interleukin-1 receptor antagonist (IL-1ra), interleukin-2 (IL-2), IL-4, IL-6, IL-8, IL-10, monocyte chemoattractant protein-1 (MCP-1/CCL2), vascular endothelial growth factor (VEGF), tissue inhibitor of metalloproteinases 1 (TIMP-1), TIMP-2, TIMP-3, TIMP-4, insulin-like growth factor 1 (IGF-1), epidermal growth factor (EGF), epidermal growth factor receptor (EGFR), fibroblast growth factors (FGF), Transforming growth factor beta 1 (TGF-31), alpha-2-microglobulin (A2M), hyaluronic acid, proteoglycans, collagen, fibronectin, laminin, trappin-2, decorin, Cystatin-c, resistin and the like, or a combination thereof.

In some embodiments, the canine-specific compositions described herein are injectable. In other embodiments, the canine-specific compositions described herein are for topical use.

In some embodiments, the canine-specific compositions described herein may be combined or mixed with stem cells, including mesenchymal stem cells. Without wishing to limit the present invention to any theory or mechanism it is believed this combination enhances stem cell stimulation, promoting increased activity and improving cell survival following injection. Stem cells offer significant therapeutic advantages in regenerative medicine, particularly due to their ability to self-renew and differentiate into various cell types. They promote tissue regeneration rather than scar tissue formation, which can lead to improved healing outcomes, especially in heart disease and muscle injuries. Their regenerative capabilities also offer long-term solutions for chronic diseases or recurring injuries, providing continuous repair and potentially slowing disease progression over time. Stem cells are especially beneficial in wound healing, accelerating the repair of skin, blood vessels, and connective tissues in difficult-to-heal wounds like diabetic ulcers and burn injuries. These properties position stem cells as a transformative tool in medicine, with the potential to significantly enhance the treatment of a broad spectrum of diseases and injuries.

In some embodiments, the canine-specific compositions described herein are combined or mixed with stromal vascular fraction.

In some embodiments, the canine-specific compositions described herein are combined or mixed with hyaluronic acid.

In some embodiments, the canine-specific compositions described herein are combined or mixed with platelet rich plasma, platelet poor plasma, or serum solution.

In some embodiments, the canine-specific compositions described herein may be administered intravenously or intra arterially. In some embodiments, the canine-specific compositions described herein may be administered intra-articularly. In some embodiments, the canine-specific compositions described herein may be administered via an intraligamentary injection and/or an intratendon injection. In some embodiments, the canine-specific compositions described herein may be administered intraoperatively. In some embodiments, the canine-specific compositions described herein may be administered by inhalation. In other embodiments, the canine-specific compositions described herein may be in the form of a liquid or gel, ointment, polymer, hydrogel, cream, lotion, foam, oil, paste, capsule, tablet, air spray or lyophilized. In some embodiments, the lyophilized canine-specific compositions described herein may be resuspended in a carrier fluid.

Methods of Treatments using the Canine-Specific Therapeutic Composition

The present invention may further feature a method of treating conditions in an animal subject. In some embodiments, the method comprises administering a therapeutic amount of the canine-specific therapeutic composition comprising a micronized canine-specific allantoamnion membrane, in a solution. In some embodiments, the composition is substantially free of blood-related contaminants and/or uteroverdin.

In some embodiments, the animal subject is a canine subject. In other embodiments, the animal subject is a feline subject.

Non-limiting conditions that may be treated by administering a canine-specific composition as described herein include but are not limited to inflammatory disease, joint disease, joint injury, meniscus injury, disk disease, hip dysplasia, tendon injury, ligament injury, muscle injury, wounds, burns, skin disease, skin dermatitis, allergies, respiratory disease, asthma, bone fracture, infections, an eye disease, eye ulcer, cataracts, glaucoma, blindness, cancer, benign tumors, organ, pain. For example, pain may include pain associated with hip dysplasia, pain associated with osteoarthritis, or pain associated with rheumatoid arthritis.

In some embodiments, the present invention may feature a method of treating cancer in a canine. The method may comprise administering a therapeutic amount of the canine-specific therapeutic composition comprising a micronized canine-specific allantoamnion membrane, in a solution. In some embodiments, the canine-specific therapeutic composition is substantially free of blood-related contaminants and/or uteroverdin. In some embodiments, the canine-specific composition is injected into a tumor. In some embodiments, the canine-specific composition is injected around a tumor. In some embodiments, the canine-specific composition is injected intravenously. In some embodiments, the canine-specific composition is injected intra arterially. In some embodiments, the canine-specific composition is administered orally. In some embodiments, the canine-specific composition is inhaled.

In other embodiments, the present invention may feature a method of preventing cancer in a canine. The method may comprise administering a therapeutic amount of the canine-specific therapeutic composition comprising a micronized canine-specific allantoamnion membrane, in a solution. In some embodiments, the canine-specific therapeutic composition is substantially free of blood-related contaminants and/or uteroverdin. In some embodiments, the canine-specific composition is injected intravenously. In some embodiments, the canine-specific composition is injected intra arterially. In some embodiments, the canine-specific composition is administered orally. In some embodiments, the canine-specific composition is inhaled.

The present invention may also feature a method of treating a benign tumor in a canine. In some embodiments, the method comprises administering a therapeutic amount of the canine-specific therapeutic composition comprising a micronized canine-specific allantoamnion membrane, in a solution. In some embodiments, the canine-specific therapeutic composition is substantially free of blood-related contaminants and/or uteroverdin. In some embodiments, the canine-specific composition is injected into a benign tumor. In some embodiments, the canine-specific composition is injected around a benign tumor. In some embodiments, the canine-specific composition is injected intravenously. In some embodiments, the canine-specific composition is injected intra arterially. In some embodiments, the canine-specific composition is administered orally. In some embodiments, the canine-specific composition is inhaled.

Additionally, the present invention may also feature a method of treating a target site. In some embodiments, the method comprises administering a therapeutic amount of the canine-specific therapeutic composition comprising a micronized canine-specific allantoamnion membrane, in a solution to the target site. In some embodiments, the canine-specific therapeutic composition is substantially free of blood-related contaminants and/or uteroverdin.

As used herein, a “target site” may refer to a treatment location. In some embodiments, the target site is an articular joint. In some embodiments, the target site is a muscle. In some embodiments, the target site is a soft tissue. In some embodiments, the target site is skin. In some embodiments, the target site is an ear. In the embodiments, the target site is the mouth. In some embodiments, the target site is an eye and ophthalmic structures. In some embodiments, the target site is the blood. In some embodiments, the target site is an artery or vein. In some embodiments, the target site is the spine (e.g., the spinal fluid). In some embodiments, the target site is an organ. In some embodiments, the target site is a bone. In some embodiments, the target site is a cartilage or the target site is a meniscus. In some embodiments, the target site is the pulmonary airways. In some embodiments, the target site is a wound. In some embodiments, the target site is an operative incision. In some embodiments, the target site is a tumor or a benign mass.

In some embodiments, the canine-specific compositions described herein may be administered via various routes, including intravenous (IV), intramuscular (IM), subcutaneous (SC), topical, or ocular, depending on factors such as the desired effects, the type of drug, the patient's condition, and other relevant considerations.

In some embodiments, 0.5 cc of the canine-specific therapeutic composition is administered to the subject. In some embodiments, 1.0 cc of the canine-specific therapeutic composition is administered to the subject. In some embodiments, 1.5 cc of the canine-specific therapeutic composition is administered to the subject. In some embodiments, 2.0 cc of the canine-specific therapeutic composition is administered to the subject. In some embodiments, 2.5 cc of the canine-specific therapeutic composition is administered to the subject. In some embodiments, 3.0 cc of the canine-specific therapeutic composition is administered to the subject. In some embodiments, 3.5 cc of the canine-specific therapeutic composition is administered to the subject. In some embodiments, 4.0 cc of the canine-specific therapeutic composition is administered to the subject. In some embodiments, 4.5 cc of the canine-specific therapeutic composition is administered to the subject. In some embodiments, 5.0 cc of the canine-specific therapeutic composition is administered to the subject. In some embodiments, 5.5 cc of the canine-specific therapeutic composition is administered to the subject. In some embodiments, 8.0 cc of the canine-specific therapeutic composition is administered to the subject. In some embodiments, 10.0 cc of the canine-specific therapeutic composition is administered to the subject. In some embodiments, 15.0 cc of the canine-specific therapeutic composition is administered to the subject. In some embodiments, 20.0 cc of the canine-specific therapeutic composition is administered to the subject. In some embodiments, 25.0 cc of the canine-specific therapeutic composition is administered to the subject. In some embodiments, 30.0 cc of the canine-specific therapeutic composition is administered to the subject. In some embodiments, 40.0 cc of the canine-specific therapeutic composition is administered to the subject. In some embodiments, 50.0 cc of the canine-specific therapeutic composition is administered to the subject. In some embodiments, 75.0 cc of the canine-specific therapeutic composition is administered to the subject. In some embodiments, 100 cc of the canine-specific therapeutic composition is administered to the subject. In some embodiments, 250 cc of the canine-specific therapeutic composition is administered to the subject. In some embodiments, 500 cc of the canine-specific therapeutic composition is administered to the subject.

The amount of the canine-specific therapeutic composition administered to the subject is not limited to the amounts mentioned above and may vary depending on the condition being treated. For instance, in the case of joint treatment, the canine-specific therapeutic composition may be administered through an injection into the subject's knee. Depending on the size of the knee, an injection of 1 cc may be sufficient to adequately fill the treatment site; however, a greater volume or smaller may be required. Alternatively, for wound treatment, the canine-specific therapeutic composition may be injected around the perimeter of the wound, thus, the volume of the composition is dependent on the size of the wound.

In some embodiments, the canine-specific therapeutic compositions described herein are used as regenerative products. In some embodiments, the canine-specific therapeutic compositions described herein are used as regenerative products to promote healing. In other embodiments, the canine-specific therapeutic compositions described herein are a biodegradable and/or bioabsorbable product. In other embodiments, the canine-specific therapeutic compositions described herein are a biodegradable and/or bioabsorbable tissue scaffold. Without wishing to limit the present invention to any theory or mechanism it is believed that after the canine-specific therapeutic composition is injected into a target site, proteins, cellular excretions, cellular derivatives, exosomes, and extracellular matrix components from the composition will elute out/be absorbed into the native surrounding tissue. Also, the membrane particles may act as a scaffold to facilitate migration of the surrounding cells, reinforce adhesion of the basal epithelium, promote cellular migration, promote cellular proliferation, promote cellular invasion, promote cellular differentiation, and prevent apoptosis.

In some embodiments, compositions described herein may be used to treat various canine ailments. Non-limiting examples of canine ailments that may be treated with compositions described herein include but are not limited to cancer, benign tumors, joint diseases, soft tissue lesions, inflammatory diseases, respiratory diseases, allergies, immunological diseases, neurological diseases, skin diseases, skin wound and repair (e.g. burns, necrosis, scarring, skin ulcers and venous ulcers), ocular wounds and repair (e.g. glaucoma, ocular ulcers, corneal ulcers, conjunctival scleral and lid and orbital rim reconstruction), coronary wounds and repair (e.g. coronary bypass, heart valve repair and replacement, vein repair and artery repair), nerve injuries, spinal injuries, muscle tears, organ diseases, bone diseases among others.

In other embodiments, compositions described herein may have anticancer effects. In other embodiments, compositions described herein may have immunoregulatory effects. In other embodiments, the compositions described herein may have anti-inflammatory effects. In other embodiments, the compositions described herein may have anti-fibrotic effects. In other embodiments, the compositions described herein may have re-epithelialization effects. In other embodiments, the compositions described herein may have antibacterial effects. In other embodiments, the compositions described herein may have antiviral effects. In other embodiments, the compositions described herein may have antifungal effects.

### Producing the Canine-Specific Therapeutic Composition

The present invention features a method of producing a canine-specific therapeutic composition. In some embodiments, the method comprises a) obtaining an allantoamnion membrane, b) micronizing said allantoamnion membrane and c) resuspending the micronized allantoamnion membrane into a solution to create a canine-specific therapeutic composition. In some embodiments, the canine-specific therapeutic composition is further lyophililzed.

In some embodiments, obtaining an allantoamnion membrane comprises obtaining a whole placenta (e.g., a whole canine placenta) and dissecting the whole placenta (e.g., a whole canine placenta) to obtain the allantoamnion membrane. The methods described herein may further comprise processing the allantoamnion membrane to retain all layers of the allantoamnion membrane. In some embodiments, the methods described herein further comprise processing the allantoamnion membrane to retain one or more layers of the allantoamnion membrane. In other embodiments, the methods described herein further comprise processing the allantoamnion membrane to retain two or more layers of the allantoamnion membrane.

In some embodiments, the methods described herein further comprise processing the allantoamnion membrane to retain at least one layer of the allantoamnion membrane. In other embodiments, the methods described herein further comprise processing the allantoamnion membrane to retain at least two layers of the allantoamnion membrane.

In some embodiments, processing the allantoamnion membrane further comprises removing the blood vessels. In some embodiments, the methods described herein further comprise rinsing to remove blood or contaminants and drying the allantoamnion membrane. In other embodiments, the methods described herein further comprise cryofracturing the allantomanion membrane to produce allantomanion particles. In other embodiments, the methods described herein further comprise resuspending the allantoamnion particles in a working solution to produce a canine-specific therapeutic composition. In other embodiments, the methods described herein further comprise freezing the canine-specific therapeutic composition. In some embodiments, freezing the canine-specific therapeutic composition preserves proteins and exosomes.

In some embodiments, the canine-specific allantoamnion membrane can be micronized using a variety of methods to achieve the desired particle size and form for specific applications. One method is cryogenic fracturing, where the membrane is subjected to extremely low temperatures to facilitate the breaking of tissue into fine particles. Additional techniques include mechanical homogenization, which utilizes homogenizers, blenders, or grinders to reduce the membrane into smaller fragments; ultrasonic disruption, wherein high-frequency ultrasonic waves generate vibrations that mechanically disrupt the tissue; and enzymatic digestion, in which enzymes such as collagenase or hyaluronidase partially digest the extracellular matrix, yielding smaller particles. Freeze-drying (lyophilization) followed by pulverization may also be employed, wherein the membrane is first freeze-dried to retain its structural integrity, and subsequently ground into microparticles. Jet milling utilizes high-velocity air or steam jets to grind the membrane into microparticles by forcing collisions between the particles. Ball milling involves placing the membrane in a rotating container with hard spheres that micronize the material through impact and friction. Electrospinning dissolves the membrane in a solvent and extrudes it through a nozzle under an electric field, resulting in micro- or nano-scale fibers or particles as the solvent evaporates. Lastly, cryo-milling combines the principles of cryogenic fracturing with high-energy ball milling under cryogenic conditions, preventing heat generation and subsequent degradation of the material. Each of these methods can be selected based on the intended application and desired characteristics of the micronized amniotic membrane, such as size, form (e.g., powder, particles, fibers), and functional properties. However, the present invention is not limited to the aforementioned methods and may also incorporate any other techniques known in the art for micronization.

The present invention may further feature a method of producing a canine-specific therapeutic composition. In some embodiments, the method comprises a) obtaining a whole canine placenta, b)dissecting the whole canine placenta and retaining an allantoamnion membrane, c) rinsing the allantoamnion membrane and drying the allantoamnion membrane, d) micronizing the aforementioned allantoamnion membrane and resuspending the micronized allantoamnion membrane into a solution to create a canine-specific therapeutic composition. In some embodiments, the method further comprises freezing the canine-specific therapeutic composition to preserve proteins and exosomes.

In some embodiments, the methods described herein further comprise lyophilization of the canine-specific therapeutic composition.

In some embodiments, the tissue (e.g., placental tissue or allantoamnion membrane) can be frozen prior to the micronizing process. The freezing step can occur by any suitable cooling process. For example, the tissue can be flash-frozen using liquid nitrogen. Alternatively, the material can be placed in an isopropanol/dry ice bath or can be flash-frozen in other coolants. Additionally, the material can be placed in a freezer and allowed to equilibrate to the storage temperature more slowly, rather than being flash-frozen. The tissue can be stored at any desired temperature. For example, −20° C. or −80° C., or other temperatures can be used for storage. In other embodiments, the methods described herein further comprises storing the canine-specific therapeutic composition at room temperature. In some embodiments, the methods described herein further comprise storing the canine-specific therapeutic composition at refrigerator temperature. In further embodiments, the methods described herein further comprise storing the canine-specific therapeutic composition below 0° C. (e.g., −20° C. or −80° C.). In further embodiments, the methods described herein further comprise storing the canine-specific therapeutic composition at room temperature.

In other embodiments, the tissue (e.g., placental tissue or allantoamnion membrane) can be decontaminated prior to or after the micronizing process. In one aspect, the premixed antibiotic solution comprising a cocktail of antibiotics such as Gentamicin and Streptomycin can be added and mixed with the tissue (e.g., placental tissue or allantoamnion membrane). In another aspect, 0.1-10% Triton-X or alcohol such as 70% isopropanol may be used. In other aspects, the tissue can be exposed to UV.

In some embodiments, the whole canine placenta is obtained after a live birth. In other embodiments, the whole canine placenta is obtained after a scheduled c-section.

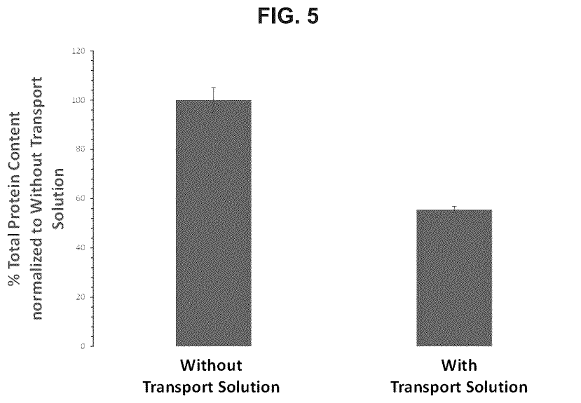

In some embodiments, the whole canine placenta is transported without a transport solution (i.e., a saline solution). In some embodiments, the whole canine placenta is transported wet. In other embodiments, the whole canine placenta is transported wet in a jar. In further embodiments, the placenta is removed from the canine and placed into a jar and then transported. Without wishing to limit the present invention to any theories or mechanisms it is believed that transporting the whole canine placenta without a transport solution (e.g., saline solution) advantageously provides for an increase in retained proteins within the placenta as compared to a canine placenta transported in a transport solution. When transported in a transport solution (e.g., a saline solution) proteins (˜40%) elute out of the placental membranes and into the transport solution (see FIG. 5). In certain embodiments, the whole canine placenta is transported in a transport solution (i.e., a saline solution).

In some embodiments, the method further comprises processing the allantoamnion membrane to retain all three layers of the allantoamnion membrane. In some embodiments, the blood vessels are removed from the allantoamnion membrane. In some embodiments, the allantoamnion membrane further comprises amniotic fluid.

In certain embodiments, the allantoamnion membrane is rinsed for 10 seconds. In other embodiments, the allantoamnion membrane is rinsed for about 5 seconds, or about 10 seconds, or about 15 seconds, or about 20 seconds, or about 25 seconds, or about 30 seconds, or about 35 seconds, or about 40 seconds, or about 45 seconds, or about 50 seconds, or about 55 seconds, or about 60 seconds, or about 65 seconds, or about 70 seconds, or about 75 seconds, or about 80 seconds, or about 85 seconds, or about 90 seconds. Without wishing to limit the present invention to any theory or mechanism it is believed that gently rinsing the allantoamnion membrane (instead of washing) allows for the retention of all three layers (i.e., a first epithelial and basal layer, a mesenchymal layer, and a second epithelial and basal layer).

In certain embodiments, the allantoamnion membrane is dried for 1 hour. In other embodiments, the allantoamnion membrane is dried for about 15 minutes, or about 30 minutes, or about 45 minutes, or about 60 minutes, or about 75 minutes, or about 90 minutes, or about 105 minutes, or about 2 hours, or about 2.25 hours, or about 2.5 hours, or about 2.75 hours, or about 3 hours. In some embodiments, the allantoamnion membrane is dried for more than 3 hours. In some embodiments, the allantoamnion membrane is air dried. In other embodiments, the allantoamnion membrane is air dried at room temperature.

In certain embodiments, the canine-specific therapeutic composition produced has a ratio of 2 cm²/mL of working solution. In other embodiments, the canine-specific therapeutic composition produced has a ratio of about 0.25 cm²/mL of working solution, or about 0.5 cm²/mL of working solution, or about 1.0 cm²/mL of working solution, or about 1.5 cm²/mL of working solution, or about 2.0 cm²/mL of working solution, or about 2.5 cm²/mL of working solution, or about 3.0 cm²/mL of working solution, or about 3.5 cm²/mL of working solution, or about 4.0 cm²/mL of working solution, or about 4.5 cm²/mL of working solution, or about 5.0 cm²/mL of working solution, or about 7.5 cm²/mL of working solution, or about 10.0 cm²/mL of working solution, or about 12.5 cm²/mL of working solution, or about 15.0 cm²/mL of working solution, or about 17.5 cm²/mL of working solution, or about 20.0 cm²/mL of working solution, or about 22.5 cm²/mL of working solution, or about 25.0 cm²/mL of working solution, or about 27.5 cm²/mL of working solution, or about 30.0 cm²/mL of working solution.

The present invention may further feature a method of treating or preventing cancer in a canine. In some embodiments, the method comprises administering a therapeutic amount of canine-specific therapeutic composition comprising placental tissue and a working solution.

### EXAMPLES

The following are non-limiting examples of the present invention. It is to be understood that said examples are not intended to limit the present invention in any way. Equivalents or substitutes are within the scope of the present invention.

### Example 1

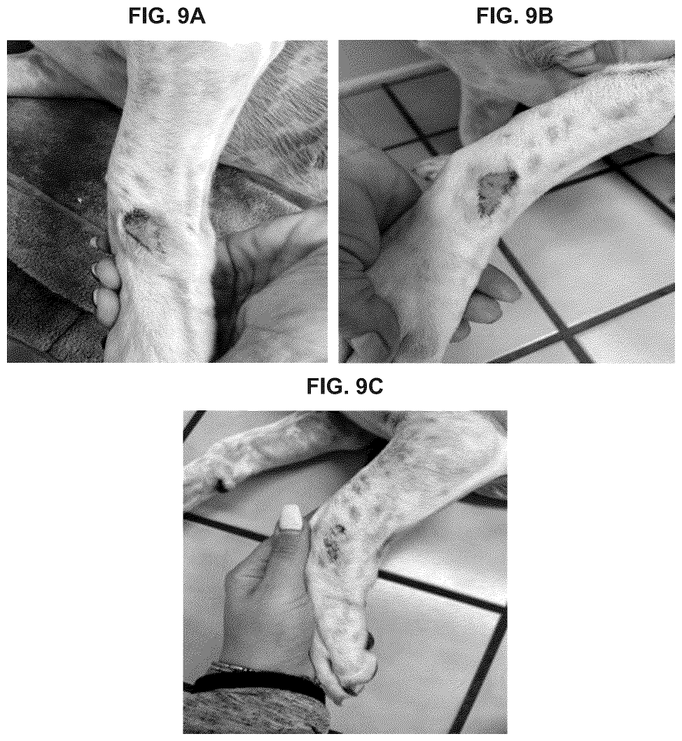

A three year-old Hound Mix dog presented with an erythematous and alopecic skin lesion on the carpal joint of the left front limb. The lesion was diagnosed as an acral lick dermatitis, and the patient received 12 rounds of laser therapy treatments. The canine-specific composition described herein was implanted subcutaneously around the margins of the wound. Two weeks after the canine-specific composition implantation, the skin lesion had reduced in size with resolving erythema (FIG. 9B). Two months following the canine-specific composition implantation, the acral lick dermatitis was resolved, and hair had grown back (FIG. 9C).

### Example 2

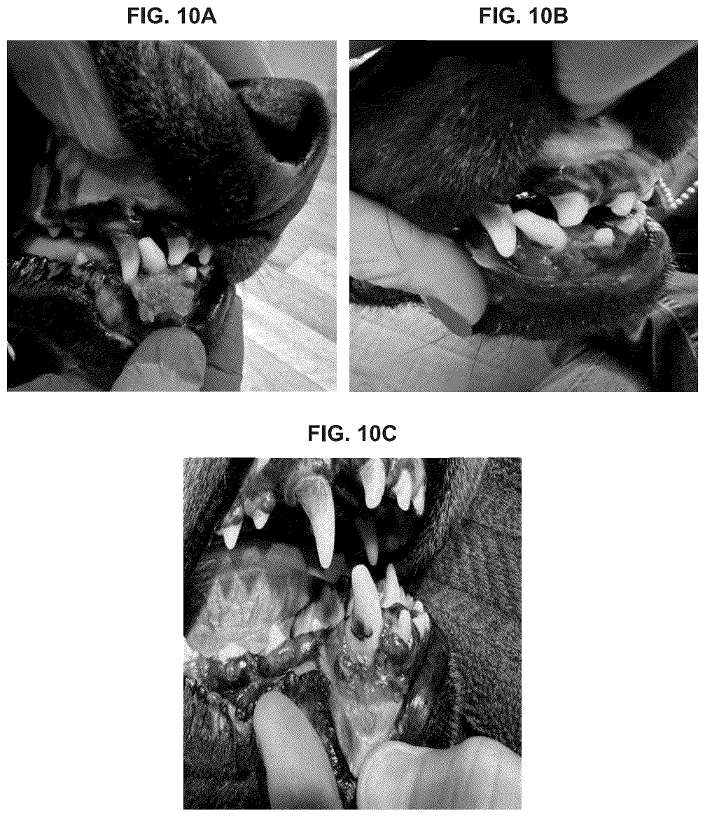

An 8 year old, spayed female Doberman Pinscher presented with a mass on the right craniolateral mandible. Biopsy samples revealed a squamous cell carcinoma. At the discretion of the veterinarian, a 2.0 cc of canine-specific composition was implanted under and around the mass without mass removal. 13 days after 2.0 cc of the canine-specific composition was implanted, the mass had significantly decreased in size compared to the initial mass (FIG. 10B). Light blunt debridement was performed using a gauze by the veterinarian. 81 Days following canine-specific composition implantation, the gum tissue looked healthy and normal at examination with no sign of recurrent mass (FIG. 10C).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

### EMBODIMENTS

The following embodiments are intended to be illustrative only and not to be limiting in any way.

Embodiment 1: A method of treating conditions in a subject, the method comprising administering a therapeutic amount of the canine-specific therapeutic composition comprising a micronized canine-specific allantoamnion membrane, in a solution.

Embodiment 2: The method of embodiment 1, wherein the composition further comprises a micronized canine-specific chorioallantois membrane, a micronized canine-specific umbilical cord, micronized canine-specific Wharton's Jelly, or a combination thereof.

Embodiment 3: The method of embodiment 1 or embodiment 2, wherein the composition further comprises micronized canine-specific allantoamnion membrane particles, micronized canine-specific chorioallantois membrane particles, micronized canine-specific umbilical cord particles, micronized canine-specific Wharton's Jelly particles, or a combination thereof.

Embodiment 4: The method of any one of embodiments 1-3, wherein the composition further comprises canine-specific amniotic fluid, canine-specific allantoic fluid, or a combination thereof.

Embodiment 5: The method of any one of embodiments 1-4, wherein the composition further comprises canine-specific amniotic fluid cells, canine-specific allantoic fluid cells, canine-specific mesenchymal stem cells, or a combination thereof.

Embodiment 6: The method of any one of embodiments 1-5, wherein the composition is substantially free of blood-related contaminants and/or uteroverdin.

Embodiment 7: The method of any one of embodiments 1-6, wherein the composition further comprises platelet poor plasma protein solution.

Embodiment 8: The method of any one of embodiments 1-7, wherein the composition further comprises serum protein solution.

Embodiment 9: The method of any one of embodiments 1-8, wherein the composition comprises proteins, wherein the proteins include one or a combination of A2M, IL-1ra, Trappin-2, IL-10, TIMP-2, MCP-1, EGFR, or VEGF. Embodiment 10: The method of embodiment 9, wherein the proteins have anti-inflammatory properties, antifibrotic properties, healing properties, repairing properties, immunoregulatory properties, anti-cancer properties, or a combination thereof.

Embodiment 11: The method of any one of embodiments 1-9, wherein the composition comprises exosomes. Embodiment 12: The method of embodiment 11, wherein the exosomes have anti-inflammatory properties, antifibrotic properties, healing properties, repairing properties, immunoregulatory properties, anti-cancer properties, or a combination thereof.

Embodiment 13: The method of any one of embodiments 1-12, wherein the composition is physiologically and biologically active.

Embodiment 14: The method of any one of embodiments 1-13, wherein the composition is mixed or combined with mesenchymal stem cells prior to administration. Embodiment 15: The method of any one of embodiments 1-13, wherein the composition is mixed or combined with platelet plasma protein solutions prior to administration.

Embodiment 16: The method of any one of embodiments 1-15, wherein the subject is a mammal. Embodiment 17: The method of any one of embodiments 1-15, wherein the subject is a canine subject.

Embodiment 18: The method of any one of embodiments 1-17, wherein the condition is an inflammatory disease. Embodiment 19: The method of any one of embodiments 1-17, wherein the condition is a joint disease. Embodiment 20: The method of any one of embodiments 1-17, wherein the condition is a joint injury. Embodiment 21: The method of any one of embodiments 1-17, wherein the condition is a disk disease. Embodiment 22: The method of any one of embodiments 1-17, wherein the condition is hip dysplasia. Embodiment 23: The method of any one of embodiments 1-17, wherein the condition is a tendon and/or ligament injury. Embodiment 24: The method of any one of embodiments 1-17, wherein the condition is a ligament injury. Embodiment 25: The method of any one of embodiments 1-17, wherein the condition is a muscle injury. Embodiment 26: The method of any one of embodiments 1-17, wherein the condition is a wound. Embodiment 27: The method of any one of embodiments 1-17, wherein the condition is a burn. Embodiment 28: The method of any one of embodiments 1-17, wherein the condition is skin dermatitis. Embodiment 29: The method of any one of embodiments 1-17, wherein the condition is allergies. Embodiment 30: The method of any one of embodiments 1-17, wherein the condition is asthma. Embodiment 31: The method of any one of embodiments 1-17, wherein the condition is a bone fracture. Embodiment 32: The method of any one of embodiments 1-17, wherein the condition is an infection. Embodiment 33: The method of any one of embodiments 1-17, wherein the condition is an eye ulcer. Embodiment 34: The method of any one of embodiments 1-17, wherein the condition is a cataract. Embodiment 35: The method of any one of embodiments 1-17, wherein the condition is glaucoma. Embodiment 36: The method of any one of embodiments 1-17, wherein the condition is blindness.

Embodiment 37: The method of any one of embodiments 1-17, wherein the condition is an organ. Embodiment 38: The method of any one of embodiments 1-17, wherein the condition is pain. Embodiment 39: The method of embodiment 38, wherein the pain is associated with hip dysplasia. Embodiment 40: The method of embodiment 38, wherein the condition is pain associated with osteoarthritis. Embodiment 41: The method of embodiment 38, wherein the pain is associated with rheumatoid arthritis.

Embodiment 42: The method of any one of embodiments 1-41, wherein the composition is administered via an injection. Embodiment 43: The method of any one of embodiments 1-41, wherein the composition is administered topically. Embodiment 44: The method of any one of embodiments 1-41, wherein the composition is administered intravenously or intraarterially. Embodiment 45: The method of any one of embodiments 1-41, wherein the composition is administered intra-articularly. Embodiment 46: The method of any one of embodiments 1-41, wherein the composition is administered via an intraligamentary injection and/or an intratendon injection.

Embodiment 47: A method of treating cancer in a subject, the method comprising administering a therapeutic amount of the canine-specific therapeutic composition comprising a micronized canine-specific allantoamnion membrane, in a solution.

Embodiment 48: A method of preventing cancer in a subject, the method comprising administering a therapeutic amount of the canine-specific therapeutic composition comprising a micronized canine-specific allantoamnion membrane, in a solution.

Embodiment 49: A method of treating a benign tumor in a subject, the method comprising administering a therapeutic amount of the canine-specific therapeutic composition comprising a micronized canine-specific allantoamnion membrane, in a solution.

Embodiment 50: The method of any one of embodiments 47-49, wherein the composition further comprises a micronized canine-specific chorioallantois membrane, a micronized canine-specific umbilical cord, micronized canine-specific Wharton's Jelly, or a combination thereof.

Embodiment 51: The method of any one of embodiments 47-50, wherein the composition further comprises micronized canine-specific allantoamnion membrane particles, micronized canine-specific chorioallantois membrane particles, micronized canine-specific umbilical cord particles, micronized canine-specific Wharton's Jelly particles, or a combination thereof.

Embodiment 52: The method of any one of embodiments 47-51, wherein the composition further comprises canine-specific amniotic fluid, canine-specific allantoic fluid, or a combination thereof.

Embodiment 53: The method of any one of embodiments 47-52, wherein the composition further comprises canine-specific amniotic fluid cells, canine-specific allantoic fluid cells, canine-specific mesenchymal stem cells, or a combination thereof.

Embodiment 54: The method of any one of embodiments 47-53, wherein the composition is substantially free of blood-related contaminants and/or uteroverdin.

Embodiment 55: The method of any one of embodiments 47-54, wherein the composition further comprises platelet poor plasma protein solution. Embodiment 56: The method of any one of embodiments 47-55, wherein the composition further comprises serum protein solution.

Embodiment 57: The method of any one of embodiments 47-56, wherein the composition comprises proteins, wherein the proteins include one or a combination of A2M, IL-1ra, Trappin-2, IL-10, TIMP-2, MCP-1, EGFR, or VEGF. Embodiment 58: The method of embodiment 57, wherein the proteins have anti-inflammatory properties, antifibrotic properties, healing properties, repairing properties, immunoregulatory properties, anti-cancer properties, or a combination thereof.

Embodiment 59: The method of any one of embodiments 47-58, wherein the composition comprises exosomes. Embodiment 60: he method of embodiment 59, wherein the exosomes have anti-inflammatory properties, antifibrotic properties, healing properties, repairing properties, immunoregulatory properties, anti-cancer properties, or a combination thereof.

Embodiment 61: The method of any one of embodiments 47-60, wherein the composition is physiologically and biologically active.

Embodiment 62: The method of any one of embodiments 47-61, wherein the composition is mixed or combined with mesenchymal stem cells prior to administration. Embodiment 63: The method of any one of embodiments 47-61, wherein the composition is mixed or combined with platelet plasma protein solutions prior to administration.

Embodiment 64: The method of any one of embodiments 47-63, wherein the subject is a mammal. Embodiment 65: The method of any one of embodiments 47-63, wherein the subject is a canine subject.

Embodiment 66: The method of any one of embodiments 47-65, wherein the composition is administered via an injection. Embodiment 67: The method of any one of embodiments 47-65, wherein the composition is administered topically. Embodiment 68: The method of any one of embodiments 47-65, wherein the composition is administered intravenously or intraarterially. Embodiment 69: The method of any one of embodiments 47-65, wherein the composition is administered intra-articularly. Embodiment 70: The method of any one of embodiments 47-65, wherein the composition is administered via an intraligamentary injection and/or an intratendon injection.

Embodiment 71: A method of treating a target site in a subject, the method comprising administering a therapeutic amount of the canine-specific therapeutic composition comprising a micronized canine-specific allantoamnion membrane, in a solution.

Embodiment 72: The method of embodiment 71, wherein the composition further comprises a micronized canine-specific chorioallantois membrane, a micronized canine-specific umbilical cord, micronized canine-specific Wharton's Jelly, or a combination thereof.

Embodiment 73: The method of embodiment 71 or embodiment 72, wherein the composition further comprises micronized canine-specific allantoamnion membrane particles, micronized canine-specific chorioallantois membrane particles, micronized canine-specific umbilical cord particles, micronized canine-specific Wharton's Jelly particles, or a combination thereof.

Embodiment 74: The method of any one of embodiments 71-73, wherein the composition further comprises canine-specific amniotic fluid, canine-specific allantoic fluid, or a combination thereof.

Embodiment 75: The method of any one of embodiments 71-74, wherein the composition further comprises canine-specific amniotic fluid cells, canine-specific allantoic fluid cells, canine-specific mesenchymal stem cells, or a combination thereof.

Embodiment 76: The method of any one of embodiments 71-75, wherein the composition is substantially free of blood-related contaminants and/or uteroverdin.

Embodiment 77: The method of any one of embodiments 71-76, wherein the composition further comprises platelet poor plasma protein solution. Embodiment 78: The method of any one of embodiments 71-77, wherein the composition further comprises serum protein solution.

Embodiment 79: The method of any one of embodiments 71-78, wherein the composition comprises proteins, wherein the proteins include one or a combination of A2M, IL-1ra, Trappin-2, IL-10, TIMP-2, MCP-1, EGFR, or VEGF. Embodiment 80: The method of embodiment 79, wherein the proteins have anti-inflammatory properties, antifibrotic properties, healing properties, repairing properties, immunoregulatory properties, anti-cancer properties, or a combination thereof.

Embodiment 81: The method of any one of embodiments 71-80, wherein the composition comprises exosomes. Embodiment 82: The method of embodiment 81, wherein the exosomes have anti-inflammatory properties, antifibrotic properties, healing properties, repairing properties, immunoregulatory properties, anti-cancer properties, or a combination thereof.

Embodiment 83: The method of any one of embodiments 71-82, wherein the composition is physiologically and biologically active.

Embodiment 84: The method of any one of embodiments 71-83, wherein the composition is mixed or combined with mesenchymal stem cells prior to administration. Embodiment 85: The method of any one of embodiments 71-83, wherein the composition is mixed or combined with platelet plasma protein solutions prior to administration.

Embodiment 86: The method of any one of embodiments 71-85, wherein the subject is a mammal. Embodiment 87: The method of any one of embodiments 71-85, wherein the subject is a canine subject.

Embodiment 88: The method of embodiment 71-87, wherein the target site/treatment location is an articular joint. Embodiment 89: The method of embodiment 71-87, wherein the target site/treatment location is a muscle. Embodiment 90: The method of embodiment 71-87, wherein the target site/treatment location is a soft tissue. Embodiment 91: The method of embodiment 71-87, wherein the target site/treatment location is the skin. Embodiment 92: The method of embodiment 71-87, wherein the target site/treatment location is the ear. Embodiment 93: The method of embodiment 71-87, wherein the target site/treatment location is the mouth. Embodiment 94: The method of embodiment 71-87, wherein the target site/treatment location is an eye and ophthalmic structures. Embodiment 95: The method of embodiment 71-87, wherein the target site/treatment location is an artery or a vein. Embodiment 96: The method of embodiment 71-87, wherein the target site/treatment location is a spinal fluid. Embodiment 97: The method of embodiment 71-87, wherein the target site/treatment location is pulmonary airways. Embodiment 98: The method of embodiment 71-87, wherein the target site/treatment location is a tumor. Embodiment 99: The method of embodiment 71-87, wherein the target site/treatment location is a benign mass. Embodiment 100: The method of embodiment 71-87, wherein the target site/treatment location is an organ. Embodiment 101: The method of embodiment 71-87, wherein the target site/treatment location is a wound or an operative incision.

Embodiment 102: The method of any one of embodiments 71-101, wherein the composition is administered via an injection. Embodiment 103: The method of any one of embodiments 71-101, wherein the composition is administered topically. Embodiment 104: The method of any one of embodiments 71-101, wherein the composition is administered intravenously or intraarterially. Embodiment 105: The method of any one of embodiments 71-101, wherein the composition is administered intra-articularly. Embodiment 106: The method of any one of embodiments 71-101, wherein the composition is administered via an intraligamentary injection and/or an intratendon injection.

Embodiment 107: A canine-specific therapeutic composition comprising a micronized canine-specific allantoamnion membrane, in a solution; wherein the composition is substantially free of blood-related contaminants and/or uteroverdin.

Embodiment 108: The composition of embodiment 107, further comprising a micronized canine-specific chorioallantois membrane, micronized canine-specific umbilical cord, or a combination thereof.

Embodiment 109: The composition of embodiment 107 or embodiment 108, further comprising micronized canine-specific allantoamnion membrane particles, micronized canine-specific chorioallantois membrane particles, micronized canine-specific umbilical cord particles, or a combination thereof.

Embodiment 110: The composition of any one of embodiments 107-109, further comprising canine-specific amniotic fluid, canine-specific allantoic fluid, or a combination thereof.

Embodiment 111: The composition of any one of embodiments 107-110, further comprising canine-specific amniotic fluid cells, canine-specific allantoic fluid cells, canine-specific mesenchymal stem cells, or a combination thereof.

Embodiment 112: The composition of any one of embodiments 107-111, wherein the composition comprises proteins, wherein the proteins have anti-inflammatory properties, antifibrotic properties, healing properties, repairing properties, immunoregulatory properties, anti-cancer properties, or a combination thereof

Embodiment 113: The composition of any one of embodiments 107-111, wherein the composition comprises exosomes, wherein the exosomes have anti-inflammatory properties, antifibrotic properties, healing properties, repairing properties, immunoregulatory properties, anti-cancer properties, or a combination thereof

Embodiment 114: The composition of any one of embodiments 107-113, wherein the composition is physiologically and biologically active.

Embodiment 115: The composition of any one of embodiments 107-114, wherein the composition is further lyophilized.

Embodiment 116: The composition of any one of embodiments 107-115, wherein the composition is mixed or combined with mesenchymal stem cells prior to administration. Embodiment 117: The composition of any one of embodiments 107-115, wherein the composition is mixed or combined with platelet plasma protein solutions prior to administration.

Embodiment 118: The composition of any one of embodiments 107-117, wherein the composition is injectable.

Embodiment 119: The composition of any one of embodiments 107-117, wherein the composition is administered topically. Embodiment 120: The composition of any one of embodiments 107-117, wherein the composition is administered intravenously or intraarterially. Embodiment 121: The composition of any one of embodiments 107-117, wherein the composition is administered intra-articularly. Embodiment 122: The composition of any one of embodiments 107-117, wherein the composition is administered via an intraligamentary injection and/or an intratendon injection.

Embodiment 123: The composition of any one of embodiments 107-122, wherein the composition is used to treat cancer, benign tumors, joint diseases, joint injuries, soft tissue lesions, inflammatory diseases, immunological diseases, neurological diseases, skin wounds, ocular wounds, coronary wounds, nerve injuries, spinal injuries, muscle tears or organ diseases in subjects in need thereof.

Embodiment 124: The composition of embodiment 123, wherein skin wounds comprise burns, necrosis, scarring, skin ulcers and venous ulcers. Embodiment 125: The composition of embodiment 123, wherein ocular wounds comprise glaucoma, ocular ulcers, corneal ulcers, conjunctival scleral or lid and orbital rim reconstruction. Embodiment 126: The composition of embodiment 123, wherein coronary wounds comprise coronary bypass, heart valve repair and replacement, vein repair and artery repair.

Embodiment 127: The composition of any one of embodiments 107-122, wherein the composition is used for skin repair, ocular repair or coronary repair.

Embodiment 128: A method of treating cancer in a canine, the method comprising administering a therapeutic amount of a composition according to any one of embodiments 107-122, to the canine in need thereof.

Embodiment 129: A method of preventing cancer in a canine, the method comprising administering a therapeutic amount of a composition according to any one of embodiments 107-122 to the canine in need thereof.

Embodiment 130: A method of producing a canine-specific therapeutic composition, the method comprising: a) obtaining or having obtained an allantoamnion membrane; b) rinsing and drying the allantoamnion membrane; c) micronizing said allantoamnion membrane; and d) resuspending the micronized allantoamnion membrane into a solution to create a canine-specific therapeutic composition.

Embodiment 131: The method of embodiment 130, wherein obtaining the allantoamnion membrane comprises obtaining a whole placenta and dissecting the whole canine placenta to obtain the allantoamnion membrane.

Embodiment 132: The method of embodiment 131, wherein the whole placenta is a whole canine placenta.

Embodiment 133: The method of any one of embodiments 130-132, further comprising processing the allantoamnion membrane to retain all layers of the allantoamnion membrane. Embodiment 134: The method of any one of embodiments 130-132, further comprising processing the allantoamnion membrane to retain one or more layers of the allantoamnion membrane. Embodiment 135: The method of any one of embodiments 130-132, further comprising processing the allantoamnion membrane to retain two or more layers of the allantoamnion membrane.

Embodiment 136: The method of any one of embodiments 130-135, wherein processing the allantoamnion membrane further comprises partially removing the blood vessels. Embodiment 137: The method of any one of embodiments 130-135, wherein processing the allantoamnion membrane further comprises removing the blood vessels.

Embodiment 138: The method of any one of embodiments 130-137, further comprising rinsing and drying the allantoamnion membrane.

Embodiment 139: The method of any one of embodiments 130-138, further comprising cryofracturing the allantomanion membrane to produce allantomanion particles.

Embodiment 140: The method of any one of embodiments 130-139, wherein the allantomanion particles are added to a working solution to produce a canine-specific therapeutic composition.

Embodiment 141: The method of any one of embodiments 130-140, further comprising freezing the canine-specific therapeutic composition.

Embodiment 142: The method of any one of embodiments 130-141, further comprising lyophilization of the canine-specific therapeutic composition.

Embodiment 143: A method of treating a joint disease in a subject, the method comprising administering a therapeutic amount of the canine-specific therapeutic composition comprising a micronized canine-specific allantoamnion membrane, in a solution.

Embodiment 144: A method of treating a joint injury in a subject, the method comprising administering a therapeutic amount of the canine-specific therapeutic composition comprising a micronized canine-specific allantoamnion membrane, in a solution.

Embodiment 145: A method of treating a soft tissue injury in a subject, the method comprising administering a therapeutic amount of the canine-specific therapeutic composition comprising a micronized canine-specific allantoamnion membrane, in a solution.

What is claimed is:

1. A method of treating conditions in a subject, the method comprising administering a therapeutic amount of the canine-specific therapeutic composition comprising a micronized canine-specific allantoamnion membrane, in a solution.

2. The method of claim 1, wherein the composition further comprises a micronized canine-specific chorioallantois membrane, micronized canine-specific umbilical cord, micronized canine-specific Wharton's jelly, or a combination thereof.

3. The method of claim 1, wherein the composition further comprises micronized canine-specific allantoamnion membrane particles, micronized canine-specific chorioallantois membrane particles, micronized canine-specific umbilical cord particles, micronized canine-specific Wharton's jelly particles, or a combination thereof.

4. The method of claim 1, wherein the composition further comprises canine-specific amniotic fluid, canine-specific allantoic fluid, canine-specific stem cells, or a combination thereof.

5. The method of claim 1, wherein the composition comprises proteins, exosomes, or a combination thereof; wherein the proteins include one or a combination of alpha-2-microglobulin (A2M), interleukin-1 receptor antagonist (IL-1ra), Trappin-2,interleukin-2 (IL-2), IL-4, IL-6, IL-8, IL-10, tissue inhibitor of metalloproteinases 1 (TIMP-1), TIMP-2, TIMP-3, TIMP-4, monocyte chemoattractant protein-1 (MCP-1/CCL2), epidermal growth factor receptor (EGFR), or vascular endothelial growth factor (VEGF).

6. The method of claim 1, wherein the composition is substantially free of blood-related contaminants and/or uteroverdin.

7. The method of claim 1, wherein the condition is an inflammatory disease or a joint disease.

8. The method of claim 1, wherein the condition is a tendon and/or ligament injury.

9. The method of claim 1, wherein the condition is pain, wherein the pain is associated with hip dysplasia or wherein pain is associated with osteoarthritis or rheumatoid arthritis.

10. The method of claim 1, wherein the composition is combined or mixed with stem cells prior to administration.

11. The method of claim 1, wherein the composition is combined or mixed with platelet plasma protein solutions prior to administration.

12. A method of treating cancer in a subject, the method comprising administering a therapeutic amount of the canine-specific therapeutic composition comprising a micronized canine-specific allantoamnion membrane, in a solution to the subject.

13. A method of treating a target site in a subject, the method comprising administering a therapeutic amount of the canine-specific therapeutic composition comprising a micronized canine-specific allantoamnion membrane, in a solution, to the target site in the subject.

14. The method of claim 13, wherein the target site is a wound or an operative incision.

15. The method of claim 13, wherein the target site is an articular joint, a muscle, or soft tissue.

16. The method of claim 13, wherein the target site is a spine or spinal fluid.

17. The method of claim 13, wherein the target site is an eye and ophthalmic structures.

18. The method of claim 13, wherein the target site is an artery or a vein.

19. The method of claim 13, wherein the target site is a tumor.

20. The method of claim 13, wherein the target site is a pulmonary airway.
